(19) United States
(12) Patent Application Publication
(10) Pub. No.: US 2004/0053228 A1
(43) Pub. Date: Mar. 18, 2004

(54) Apoptosis inhibitory polypeptides, gene and polynucleotide encoding it and compositions containing the same

(76) Inventors: Futoshi Shibazaki, Tokyo (JP); Hidekazu Kuma, Ibaraki (JP)

Correspondence Address: FITCH EVEN TABIN AND FLANNERY, 120 SOUTH LA SALLE STREET, SUITE 1600, CHICAGO, IL, 60603-3406, US

(21) Appl. No.: 10/148,953

(22) PCT Filed: Dec. 7, 2000
(86) PCT No.: PCT/JP00/08667

(30) Foreign Application Priority Data
Dec. 9, 1999 (JP) 11-350427

(51) Int. Cl. C07K 14/47; C12N 15/12; C07H 21/04; C12Q 1/68

(52) U.S. Cl. 435/6; 435/69.1; 435/320.1; 435/325; 530/350; 536/23.5

(57) ABSTRACT

At least one serine in the amino acid sequence of the wild type Bcl-2 protein is substituted by alanine or asparagic acid to yield a polypeptide wherein the apoptosis inhibitory activity of the wild type Bcl-2 protein has been enhanced.

### TECHNICAL FIELD

This invention relates to genes regulating the apoptosis of cells. More particularly, the invention relates to the variants of oncogene Bcl-2 derived from human follicular B cell lymphoma and to the variants of the apoptosis inhibitory Bcl-2 proteins encoded by the former variants.

### BACKGROUND ART

1. Cell Death and Apoptosis

In the past, for cell death, only the “necrosis” was known which was the pathological cell death where a group of cells died of burn or the like. In 1972, the three pathologists, Kerr, Wyllie and Currie discovered apoptosis and defined that it was the cell death which differs from necrosis in morphological aspects (Kerr et al., Br, J. Cancer 26, 239 (1972)). It was proposed that apoptosis was different from necrosis not only in morphology, but also in function and that apoptosis had broad significance in the cell kinetics of tissue. Because of its characteristics, it has been regarded as the active process programmed in genes (programmed cell death).

Apoptosis is a process by which many cells die for purposes of the growth and maintenance of the cells themselves in complex eucaryotes. The cell death by apoptosis occurs when the cell activates its suicide program coded within through exogenous or endogenous signals. Apoptosis is characterized by the blebbing of cell membrane, the loss in cell volume, karyopyknosis (nuclear condensation) and the decomposition of DNA at nucleosome spaces (Wyllie et al., Int. Rev. Cytol. 68, 251 (1980)).

Apoptosis plays an indispensable role in the maintenance of function of the normal body from the early phase of the birth of life beginning with one fertilized egg to death; and its aberration is involved in a number of diseases including carcinoma. It has been revealed that common genes participate in the mechanism of such cell death. In addition, it is beginning to be understood that many of oncogenes and cancer-suppressing genes are related to apoptosis. Apoptosis is also seen in the manifestation of physiological phenomena such as immunity and hormonal action and it plays an important role as “physiological cell death” which is essential to these life phenomena. Recent studies on the mechanism for regulating the programmed cell death are advancing very rapidly (Williams, Cell 65, 1097 (1991)).

2. Aberration of Apoptosis, Disease and Therapy

The immune system central to biological defense mechanism is the living system that is most closely related to apoptosis. In the development of the immune system centered on the thymus, the aberration of apoptosis-associated genes causes an autoimmune disease. In viral infections such as HIV, apoptosis may induce abrupt reduction in lymphocytes. Apoptosis also plays an important role in the development of nervous system. In addition, apoptosis is regarded as important in the disorders involving nerve cell death such as Alzheimer disease, Parkinson disease, and dysmnesia resulting from reperfusion damage. Involvement of apoptosis in the pathology of carcinogenesis has also caught much attention. As compared with the high frequency of carcinogenesis in the large intestine, the frequency of carcinogenesis in the small intestine is extremely low: the high occurrence of apoptosis in the intestinum tenue epithelia stem cell accounts for it. Thus, the possibility can be suggested that apoptosis is directly involved in the pathology of carcinogenesis.

In addition to the etiologic investigation as described above, a new thought is going to be created in the field of therapy. So far only the growth inhibition of cancer cells has been the target of treatment in cancer therapy, but an approach is coming out that the apoptosis of the cancer cells is actively induced to enhance therapeutic effects.

In this way, since too much or too little in cell death is involved in many diseases, expectation in the therapy by the control of apoptosis is rising. Accordingly, the possibility will be opened up that the development of factors or drugs which promote or inhibit apoptosis can be applied to the therapy of diseases associated with apoptosis.

3. Apoptosis-Associated Genes (Bcl-2 gene)

In 1985 Tsujimoto et al. discovered Bcl-2 gene as an oncogene that was located in the neighborhood of the t (14; 18) (q32; q21) translocation point which was found with high frequency in human follicular B cell lymphoma (Tsujimoto Y. et al, Science 228, 1440 (1985)). Since Vaux et al. reported the apoptosis inhibitory effect of Bcl-2 in 1988, the protective effect of Bcl-2 has been shown in many systems where apoptosis is induced. Searches for the genes homologous to the Bcl-2 as well as for factors binding to the Bcl-2 protein have been enthusiastically conducted, and a large number of family genes have been identified since 1993.

The Bcl-2 gene consists of three exons. The Bcl-2 gene generates plural mRNAs depending on the presence of splicing, and the production of two kinds of proteins (Bcl-2α with 26 kD and Bcl-2β with 22 kD) is expected; however, only Bcl-2α has been detected in the living body. The Bcl-2 protein has a hydrophobic amino acid domain at its C-terminal which serves as a membrane localization signal and is present in extranuclear membrane, endoplasmic reticulum membrane, and mitochondrial membrane.

It has been shown that Bcl-2 can efficiently inhibit the apoptosis induced by almost any stimuli and it is thought that the Bcl-2 functions in the common path of apoptosis with Bcl-XL. on the other hand, Bax, Bak, Bad, Bik, and Bcl-XS, which are other members of the Bcl-2 family, suppress the inhibitory effect of Bcl-2 or Bcl-XL. It is thought that this function of Bax and the others can be achieved by forming a heterodimer with Bcl-2 or with Bcl-XL. It has been reported that Bcl-2 can bind to R-Ras, Bag-l, and Raf-1 besides the family members mentioned above. In addition, Bcl-2 and Bcl-XL can suppress some necrosis (through mitochondrial respiratory chain inhibition).

The transgenic mouse that overexpresses the Bcl-2 in the B cell system exhibits benign lymphadenopathy and then develops a malignant lymphoma with constant frequency; in addition, the crisis of an autoimmune disease resembling SLE (systemic lupus erythematodes) is observed. The transgenic mouse that overexpresses the Bcl-2 in T cells does not exhibit excessive T cell accumulation. It is shown that the removal of autoantigen recognition T cells from T cells occurred in the thymus progresses almost normally.

In the transgenic mouse where a high level expression of Bcl-2 in nerve cells is caused, the removal of the excessive nerve cells generated during the normal ontogenesis is suppressed. This mouse also shows resistance to cell death by denervation or ischemia. In the transgenic mouse where a high level expression of Bcl-2 in hepatic cells is caused, the induction of fulminant hepatitis by anti-Fas antibody is significantly suppressed.

A Bcl-2 defect mouse is born normally at a glance, but is accompanied by aberration due to a short cycle of lymphocytes and intestinal epithelium in addition to aberration such as growth retardation, little external ear pinna, canities, and multicystic kidney. In thymus T cells, the expression of Bcl-2 is observed in a mature T cell (CD4+CD8− and CD4−CD8+), and its expression in an immature T cell (CD4+CD8) is low. It is shown that the Bcl-2 plays an important role in the maintenance of mature thymocytes.

4. Phosphorylation of the Bcl-2 Gene

Haldar et al. pointed out the possibility that the Bcl-2 lost its apoptosis inhibitory effect through Bcl-2 phosphorylation (Haldar S. et al., PNAS 92, 4507 (1995)). They found that part of serine residues in the Bcl-2 underwent phosphorylation in lymphocytes and showed that the anti-apoptosis ability of the Bcl-2 was inhibited in the system okadaic acid and taxol were added which were hydrolase inhibitors of phosphoric acid.

In addition, Shibasaki found that calcineurin, which is a dephosphorylation enzyme of serine/threonine bound to the BH-4 domain (Shibasaki F. et al., Nature 386, 728-731 (1997)). They showed that calcineurin, which is a dephosphorylation enzyme of serine/threonine regulated phosphorylation/dephosphorylation and controlled the function of the Bcl-2 by directly binding to the BH-4 domain of Bcl-2.

### DISCLOSURE OF THE INVENTION

This invention aims at providing Bcl-2 variant proteins possessing more potent apoptosis inhibitory activity than does the wild type Bcl-2, as well as providing Bcl-2 variant genes encoding the proteins.

As stated above, it is anticipated that the phosphorylation/dephosphorylation of serine residues in the Bcl-2 molecule influences the apoptosis inhibitory activity. Accordingly, the present inventors focused the importance of these serine residues and carried out studies diligently to improve the apoptosis inhibitory activity of the wild type Bcl-2; consequently, it was discovered that the variants where alanine or asparagic acid substituted for the 24th serine existing in the BH-4 domain displayed significantly enhanced apoptosis inhibitory activity as compared with the wild type Bcl-2.

The present inventors also discovered that the variants where alanine or asparagic acid substituted for a particular position(s) existing other than in the BH-4 domain displayed significantly enhanced apoptosis inhibitory activity as compared with the wild type Bcl-2; and this invention was thus accomplished.

In other words, the gist of this invention is that by substituting alanine or asparagic acid for serine among amino acids constituting the wild type Bcl-2 protein to enhance its apoptosis inhibitory activity, the resulting variant proteins or the variant genes encoding them are used for the treatment of various diseases in which apoptosis is involved and the therapeutic effects are to be achieved based on the enhanced apoptosis inhibitory effect.

Consequently, there is provided according to this invention a polypeptide comprising an amino acid sequence derivable from the substitution of at least one serine by alanine or asparagic acid in the amino sequence set forth in SEQ ID NO:1 in the Sequence Listing, said polypeptide possessing apoptosis inhibitory activity.

Preferably, there is provided according to this invention a polypeptide comprising an amino acid sequence derivable from the substitution of at least one serine by alanine or asparagic acid in the amino sequence set forth in SEQ ID NO:1 in the Sequence Listing, said polypeptide possessing apoptosis inhibitory activity substantially higher than that of the wild type Bcl-2 protein.

Preferably, serine is at least one selected from the 24th residue, the 116th residue, the 117th residue or the 161st residue of the amino acid sequence in the polypeptide mentioned above.

More preferably, the invention is characterized in that the 24th, the 116th and/or the 117th serine is substituted by alanine, or the 24th, the 116th and/or the 161st serine is substituted by alanine or asparagic acid in the polypeptide mentioned above.

Specifically, this invention provides the proteins described in 1-4 below:

1. A polypeptide comprising the amino acid sequence set forth in SEQ ID NO:2 in the Sequence Listing.

2. A polypeptide comprising the amino acid sequence set forth in SEQ ID NO:3 in the Sequence Listing.

3. A polypeptide comprising the amino acid sequence set forth in SEQ ID NO:4 in the Sequence Listing.

4. A polypeptide comprising the amino acid sequence set forth in SEQ ID NO:5 in the Sequence Listing.

Any of the polypeptides described in 1-4 above is a variant of the wild type Bcl-2 protein and is provided with the apoptosis inhibitory activity substantially higher that that of said protein.

Furthermore, this invention provides a pharmaceutical composition comprising any one of the polypeptides possessing the apoptosis inhibitory activity as described above, a combination thereof, or a partial peptide thereof, together with a pharmaceutically acceptable carrier or diluent. Here, the polypeptide or the partial peptide may be in the form of its respective pharmaceutically acceptable salts.

This invention also provides a gene or its corresponding polynucleotide which gene encodes any one of the aforementioned polypeptides possessing the apoptosis inhibitory activity.

More specifically, this invention provides the polynucleotides described in 5-8 below:

5. A polynucleotide comprising the nucleotide sequence set forth in SEQ ID NO:7 in the Sequence Listing.

6. A polynucleotide comprising the nucleotide sequence set forth in SEQ ID NO:8 in the Sequence Listing.

7. A polynucleotide comprising the nucleotide sequence set forth in SEQ ID NO:9 in the Sequence Listing.

8. A polynucleotide comprising the nucleotide sequence set forth in SEQ ID NO:10 in the Sequence Listing.

Any of the polynucleotides described in 5-8 above is a nucleotide variant of the wild type Bcl-2 gene and the corresponding gene encodes the variant of the wild type Bcl-2 protein mentioned above.

This invention further provides a viral vector having the aforementioned gene incorporated in the state capable of being expressed.

In addition, this invention provides a composition for gene therapy comprising any one of the polynucleotides mentioned above and a pharmaceutically acceptable carrier or diluent.

As used in the specification, the terms, “Bcl-2” and “Bcl-2 protein” are used interchangeably as appropriate. In addition, the “Bcl-2” means “the wild type Bcl-2 protein” unless stated otherwise.

### BRIEF DESCRIPTION OF THE DRAWINGS

FIG. 1 is a schematic structural representation of Bcl-2 showing the relative positional relation between each domain of the wild type Bcl-2 type protein and each serine residue to be substituted upon the introduction of variation.

FIG. 2 is a schematic diagram showing the scheme in which plasmids containing a Bcl-2 variant gene are constructed by the techniques including PCR, restriction enzyme digestions, and site-specific mutation according to this invention.

FIG. 3 is a bar graph comparing the wild type Bcl-2 protein to various kinds of Bcl-2 variants obtained by introducing one or more variations to said protein according to this invention with respect to the apoptosis inhibitory activity.

### BEST MODE FOR CARRYING OUT THE INVENTION

This invention will be described in detail hereafter by referring to preferred embodiments.

(Bcl-2 Variant Proteins and Bcl-2 Variant Genes)

The full amino acid sequence of Bcl-2 and the full nucleotide sequence of its corresponding Bcl-2 gene are known in the art and are shown in SEQ ID NO:1 and NO:6 in the Sequence Listing, respectively. The polypeptide of this invention is a Bcl-2 variant protein and comprises an amino acid sequence derivable from the substitution of at least one serine in the amino acid sequence of Bcl-2 (SEQ ID NO:1) by alanine or asparagic acid. Preferably, the polypeptides of this invention have the apoptosis inhibitory activity substantially higher than that of the Bcl-2 protein.

Especially preferred individual variant proteins are described in 1-4 below.

1. Ser24Ala Variant

It is a variant derivable from the substitution of the 24th serine by alanine in the amino acid sequence of Bcl-2, and is a polypeptide the amino sequence of which is set forth in SEQ ID NO:2. The gene encoding this variant is represented by the polynucleotide comprising the nucleotide sequence set forth in SEQ ID NO:7.

2.Ser116Ala/Ser117Ala Variant

It is a variant derivable from the substitution of the 116th serine by alanine and of the 117th serine by alanine in the amino acid sequence of Bcl-2, and is a polypeptide the amino acid sequence of which is set forth in SEQ ID NO:3. The gene encoding this variant is represented by the polynucleotide comprising the nucleotide sequence set forth in SEQ ID NO:8.

3. Ser161Ala Variant

It is a variant derivable from the substitution of the 161st serine by alanine in the amino acid sequence of Bcl-2, and is a polypeptide the amino acid sequence of which is set forth in SEQ ID NO:4. The gene encoding this variant is represented by the polynucleotide comprising the nucleotide sequence set forth in SEQ ID NO:9.

4. Ser161Asp Variant

It is a variant derivable from the substitution of the 161st serine by asparagic acid in the amino acid sequence of Bcl-2, and is a polypeptide the amino sequence of which is set forth in SEQ ID NO:5. The gene encoding this variant is represented by the polynucleotide comprising the nucleotide sequence set forth in SEQ ID NO:10.

Bcl-2 variants other than those described in 1-4 mentioned above and the genes encoding them are encompassed by this invention. According to the disclosure of this specification, a Bcl-2 variant is prepared, and its apoptosis inhibitory activity is, for example, determined based on the screening panel described in the examples. When the determined apoptosis inhibitory activity is higher than that of the wild type Bcl-2, the Bcl-2 variant is within the scope of the polypeptide of this invention. According to the aforesaid disclosure and the techniques known in the art, one skilled in the art can carry out this series of manipulations without attempting undue experimentation.

The polypeptides of this invention are “substantially pure”; and as is recognized by one skilled in the protein filed, it means that when the polypeptide is produced by genetic manipulation, the polypeptide is free from contamination by proteins, nucleic acids, and other biological substances derived from the host organism. Further, it means that when the polypeptide is chemically produced by the solid phase peptide synthesis, the polypeptide is free from contamination by impurities such as the reagents used in the synthesis. Furthermore, the polypeptides of this invention encompass fragments of the Bcl-2 variants defined above; part of the peptide as described in any of 1-4. Such a fragment can be used to readily produce an antibody specifically binding to the polypeptide of this invention. The antibody is useful for the purification of the polypeptide of this invention, particularly using affinity chromatography.

(Method of Production)

The polypeptide of this invention comprises an amino acid sequence derivable from the substitution of at least one serine by alanine or asparagic acid in the amino acid sequence of Bcl-2. Once the location of serine to be substituted for is specified, the polypeptide of this invention can be produced from the Bcl-2 variant gene. For such a purpose, it is site-specific mutagenesis that is employed most commonly as the variation method of nucleotide sequence in gene. For example, M13 primer mutation, PCR and other improved methods are known. “Sambrook et al., Molecular Cloning: A Laboratory Manual (2nd Ed) Vol. 2, 1989” is referred to as a general review. If the synthetic oligonucleotide into which the desired variation has been introduced is available, the predetermined amino acid of the wild type protein to be desirably varied can be substituted by the amino acid that the variation encodes.

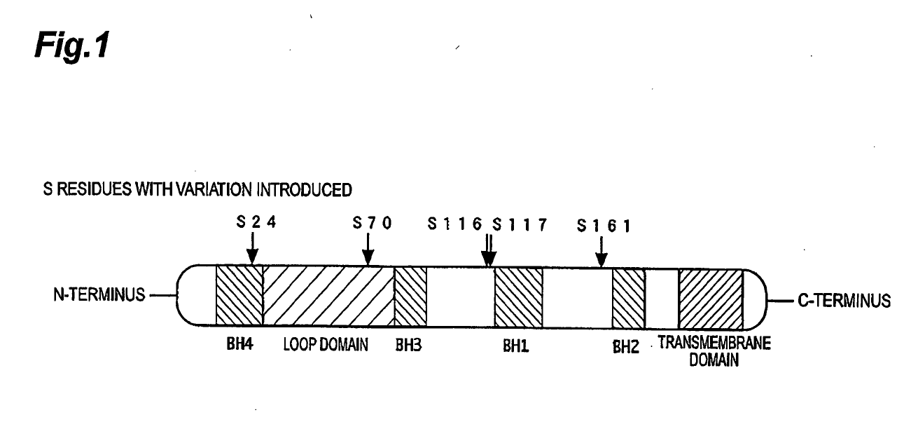

The locations of particular serine residues to be substituted for through variation as employed in the embodiments of this invention are at least one location selected from the 24th, the 70th, the 116th, the 117th or the 161st position in the amino acid sequence of Bcl-2. The particularly preferred is at least one location selected from the 24th, the 116th, the 117th or the 161st position. In consideration of these preferable locations, the preferable pattern of variation is a variation of such type that the 24th, the 116th, and/or the 117th serine are substituted by alanine and/or the 161st serine is substituted by alanine or asparagic acid. FIG. 1 shows the relative positional relation between these substitution (variation) locations and each domain of Bcl-2. To substitute these locations with alanine or asparagic acid, synthetic oligonucleotides illustrated below were prepared.

Oligo-1: oligonucleotide for introducing S24A variation (SEQ ID NO: 11) 5′-gtagccggtctgcgccagcttataatg-3′

Oligo-2: oligonucleotide for introducing S24D variation (SEQ ID NO:12) 5′-gtagccggtctggtccagcttataatg-3′

Oligo-3: oligonucleotide for introducing S70A variation (SEQ ID NO:13) 5′-ggtcttgcagcggcgcggtcctggcgc-3′

Oligo-4: oligonucleotide for introducing S70D variation (SEQ ID NO:14) 5′-ggtctgcagcgggtcggtcctggcgac-3′

Oligo-5: oligonucleotide for introducing S116A variation (SEQ ID NO;15) 5′-gtgcagctggctggccatctcggcgaa-3′

Oligo-6: oligonucleotide for introducing S116D variation (SEQ ID NO:16) 5′-gtgcagctggctgaccatctcggcgaa-3′

Oligo-7: oligonucleotide for introducing S116A/S117A variation (SEQ ID NO:17) 5′-gtgcagctgggcggccatctcggcgaa-3′

Oligo-8: oligonucleotide for introducing S161A variation (SEQ ID NO:18) 5′-ctcccggttgacggcctccacacacat-3′

Oligo-9: oligonucleotide for introducing S161D variation (SEQ ID NO:19) 5′-ctcccggttgacgacctccacacacat-3′

According to the one letter designation for amino acids, “S” refers to serine, “A” to alanine, and “D” to asparagic acid in the above description.

As for the length of a synthetic oligonucleotide, it is not limited to the length adopted in the specific disclosed examples above if it is sufficient to hybridize to the wild type gene at the predetermined site to be subjected to variation.

Although a particular site-specific mutation method is described in detail in the Examples, it will be carried out by the procedure in what follows. When the synthetic oligonucleotide is treated with a kit for site-specific mutation together with a suitable plasmid into which the Bcl-2 gene is incorporated, said oligonucleotide hybridizes to the predetermined site of the gene as the template DNA under annealing conditions. This is subjected to the action of T4 DNA polymerase in the presence of T4 ligase, which results in a plasmid having the gene which contain the desired variation. The gene having undergone variation is cut out with restriction enzyme.

The thus obtained Bcl-2 variant gene is linked to a suitable vector/promoter and transferred to a host cell system, and the protein encoded by the gene is allowed to be produced, from which the peptide of this invention can be obtained by extraction and purification. These manipulations are known to one skilled in the art. The vector/promoter system to be is not particularly limited. Specifically, there are mentioned viral vectors, including MoMLV vector, HSV vector, Adenovirus vector, AAV vector, HIV vector, SlV vector, and Sendai virus vector. Usable are also a baculovirus vector capable of gene transfer to insect cells and a vector derived from tobacco mosaic virus capable of gene transfer to plant cells

As for the vectors other than those of viral origin, there may be used complexes of calcium phosphate and nucleic acid, ribosomes, cation-lipid complexes, Seidai virus liposomes, polymer carriers having polycation as the main chain and others. In addition, methods such as electroporation and gene guns may be used in gene transfer.

The promoters are not particularly limited insofar as they can allow the genes to be expressed in the host cells. Specifically, there may be mentioned: virus-derived promoters from Adenovirus, cytomegalovirus, human immunodeficiency virus, simian virus 40, Rous sarcoma virus, herpes simplex virus, murine leukemia virus, Sinbis virus, hepatitis type A virus, hepatitis type B virus, hepatitis type C virus, papilloma virus, human T cell leukemia virus, influenza virus, Japanese encephalitis virus, JC virus, parbovirus B19, poliovirus, and the like; mammal-derived promoters such as albumin, SR α, heat shock protein, and elongation factor; chimera type promoters such as CAG promoter; and promoters the expression of which can be induced by tetracycline, steroids, or the like. There may be also used promoters (such as Lac promoter) capable of expression in E. coli host cells.

The host cell system includes animal cells, insect cells, plant cells, E. coli and embryonated chicken eggs, for example.

Furthermore, with respect to the method for extracting the protein produced by the gene transferred host cell system, there are the cell homogenization method, the cell membrane lysis method utilizing a surfactant (e.g., SDS) or enzyme, ultrasonication, the freeze and thaw repetition method among others. The polypeptides of this invention can be produced by chemical synthesis using a commercially available peptide synthesizer besides gene manipulation techniques mentioned above, because their amino acid chain lengths are comparatively short. The polypeptides of this invention obtained by any of the methods may be purified according to conventional methods. Specifically, typical biochemical techniques are available for the purification which include centrifugation utilizing ultracentrifugation or density gradient centrifugation, column separation utilizing ion-exchange column or affinity column (e.g., with use of the aforementioned specific antibodies), reversed-phase column, and gel separation utilizing polyacrylamide gel.

(Pharmaceutical Compositions)

The polypeptides that are produced and purified as described above may be combined with pharmaceutically acceptable carriers or diluents and may be formulated into pharmaceutically useful compositions. The pharmaceutical composition used in this invention comprises a therapeutically effective amount of one or more polypeptides mentioned above. For the suitable carriers and diluents as well as preparations containing human proteins, there are described in Remington's Pharmaceutical Sciences, for example.

The polypeptide of this invention may be formulated into a pharmaceutical composition in the form of a pharmaceutically acceptable salt. Such pharmaceutically acceptable salts include those formed with the free amino groups of protein such as those derived from hydrochloric acid, phosphoric acid, acetic acid, oxalic acid and tartaric acid and those formed with the free carboxylic acids of protein such as those derived from sodium, potassium, ammonium, calcium, ferric hydroxide, isopropylamine, triethylamine, 2-ethylaminoethanol, histidine, and procaine.

Suitable dosage forms adapted to administration for the polypeptides of this invention are not particularly limited, and preferably, they may be prepared into injectables similarly to many pharmaceutical compositions containing human proteins already for use as medicines. More specifically, the polypeptide is dissolved in a suitable solvent such as water, a physiological saline solution, and an isotonization buffer to make an injectable. Here, polyethylene glycol, glucose, various amino acids, collagen, albumen or the like may be added as protection materials for preparation. In addition, the polypeptide can be embedded into the inclusion body of ribosome and can be administered.

When the polypeptide of this invention is used in the treatment of various diseases such as those mentioned below, the dosage differs depending on the age of the subject, the body weight, the conditions, the route of administration and other factors; but the prescribing physician can readily and appropriately determine it. For example, when it is administered through parenteral administration as an injectable, the administration with a daily dose of about 0.1 μg/kg-1000 mg/kg is preferable, and more preferably with a daily dose of about 1 μg/kg-100 mg/kg.

(Gene Therapy)

The polynucleotides of this invention can be used as therapeutic genes (drug genes) in gene therapy. Specifically, the polynucleotide of this invention is inserted into a recombinant vector (gene transfer vector) and is delivered to the target cell, where the Bcl-2 variant gene is expressed. Then the polypeptide of this invention (i.e., a specific Bcl-2 variant) is produced in the target cell and inhibit the apoptosis of said cell.

The suitable recombinant vectors to be used for this purpose overlap the vectors capable of gene transfer to mammals, particularly human cells among those recited. Among vectors that are not disclosed above, there is mentioned a pseudo-type viral vector, for example. One representative is the pseudo-type viral vector wherein the Env protein (an envelop protein of HIV) is substituted with the VSV-G protein (an envelop protein of vesicular stomatitis virus or VSV) (Naldini L., et al., Science 272, 263 (1996)).

Likewise, the usable promoters overlap the vectors capable of gene expression in mammal cells, particularly human cells among those recited.

(Compositions for Gene Therapy)

When the polynucleotide of this invention is used in gene therapy, it is preferably prepared as a recombinant viral vector containing a therapeutic gene designed for therapy. More specifically, the recombinant viral vector containing the polynucleotide of this invention is dissolved in a suitable solvent such as water, a physiological saline, or an isotonization buffer to prepare a composition for gene therapy.

(Utility)

A. Therapy

Apoptosis is involved in various disorders, and by inhibiting cell death through apoptosis (degeneration), therapeutic effects can be expected for those disorders. Accordingly, the polypeptides and polynucleotides according to this invention (which may be referred to as “Bcl-2 variant” and “Bcl-2 variant gene” hereafter) can be used for the therapy of those disorders because of their inhibitory activity. Representative disorders will be listed individually in what follows.

1. Alzheimer Disease

Alzheimer disease is an encephalo-degenerative disorder of which progressive dysmnesia and intelligence degradation are the chief symptoms and it is known that the degeneration death of neurons causes its crisis. It has been recently understood that this cell death is induced by apoptosis. Accordingly, therapeutic effects can be expected for the treatment of Alzheimer disease by introducing the Bcl-2 variant or the Bcl-2 variant gene into the neurons.

2. Amyotrophic Lateral Sclerosis (ALS)

Amyotrophic lateral sclerosis (ALS) is an encephalo-degenerative disorder of which myodynamia depression at the distal parts of the limb, muscular atrophy, and dysphagy are the chief symptoms, and lower motor neurons are progressively and selectively damaged. Although the details of molecular biological mechanism of the crisis are unknown, it is becoming clear that apoptosis is induced by the neurons owing to gene abnormalities such as superoxide dismutase (SOD) and nerve growth nutritional factor (BDNF). Accordingly, therapeutic effects can be expected for the treatment of amyotrophic lateral sclerosis by introducing the Bcl-2 variant or the Bcl-2 variant gene into the neurons.

3. Nerve Cell Death After Cerebral Ischemia

Nerve cell death occurs in the hippocampus CA1 region after cerebral ischemia or cerebral infarction, which in turn proves to be the cause for dysmnesia and neurodegeneration disorder. It has recently been understood that this cell death is induced by apoptosis. Accordingly, therapeutic effects can be expected for the treatment of nerve cell death after cerebral ischemia by introducing the Bcl-2 variant or the Bcl-2 variant gene into the neurons.

4. Retinal Degenerative Disorders

In the retinal degenerative disorder, nyctalopia develops from the latter half of teens to the latter half of the 20's and then afferent narrowing of visual field progresses, leading to blindness during between 40's and 60's in many cases. It is known that these symptoms are caused by the cell degeneration of retina visual cells through apoptosis. Accordingly, therapeutic effects can be expected for the treatment of retinal degenerative disorder by introducing the Bcl-2 variant or the Bcl-2 variant gene into the retinal visual cells.

5. Hepatic Disorders

Hepatic disorders such as hepatitis and hepatic insufficiency develop by the cell death of hepatocytes; and this cell death is known to be apoptosis. Accordingly, therapeutic effects can be expected for the treatment of hepatic disorders by introducing the Bcl-2 variant or the Bcl-2 variant gene into the hepatocystes.

6. Heart Failure

When rat myocardial cells are cultured under anoxia in a myocardial ischemia model test, DNA laddering or an increase of Fas antigen are observed, and the cell death by apoptosis is generated. The possibility is suggested that the apoptosis of such a myocardial cell participates in heart failure such as myocardial infarction, heart failure, and hypertrophic heart. Accordingly, therapeutic effects can be expected for the treatment of heart failure by introducing the Bcl-2 variant or the Bcl-2 variant gene into the myocardial cells.

B Other Utilities

The polypeptides and the polynucleotides according to this invention can be used for the purpose of inhibiting the cell death through apoptosis in addition to the therapy of diseases.

1. Production Cell Lines

The cell lines capable of producing useful proteins such as antibody producing cell lines (hybridomas) and viral vector producing cell lines may sometimes causes cell death because of the cytotoxicity of the protein produced while the lines are cultured for a long period of time. The production of protein can be carried out more efficiently in these cell lines by removing blood serum from the culture medium, but there is the problem that the cell death is induced in the absence of the blood serum at the same time. The major part of cell death is known to be due to apoptosis. There has been obtained the information that the production efficiency of protein increases in the strain into which the wild type Bcl-2 is incorporated (WPI97-380167/199735). It is thus expected that the production efficiency of protein increases more by the Bcl-2 variant gene transfer.

2. Graft Cells and Transplants

In the case of Parkinson disease's therapy, the technique is used that transplants the nerve cells of a dead fetus or dopamine-producing cells in the brain of a patient. Further, blood transfusion, bone marrow transplantation, and cell transplantation therapy which introduces autologous cells or the cells of others into a patient, including the ex vivo method in gene therapy, have been conducted in many instances. Graft cells typically undergo cell death by apoptosis and their long-term maintenance is difficult. Effects can be expected in organ transplantation and cell transplantation by introducing the Bcl-2 variant or the Bcl-2 variant gene into the graft cells.

### EXAMPLES

This invention will be described more concretely by way of examples; however, the invention is not to be limited by these examples.

### Example 1

### Construction of Bcl-2 Variant Genes

The human wild type Bcl-2 gene was provided by S. Korsmeyer at Washington University. There were synthesized a primer having an XhoI site (which was a restriction enzyme site) just prior to the transcription start codon of the human wild type Bcl-2 (Oligo-10: SEQ ID NO:20) and a primer having an XbaI site just after the transcription stop codon (Oligo-11: SEQ ID NO:21), respectively. PCR was performed in the procedure described below.

Human Bcl-2 gene, 1 μl, dNTP (2 nM: Promega Corporation), 5 μl, Oligo-10 (50 μM), 1 μl, Oligo-11 (50 μM), 1 μl, polymerase (2-3 units: Promega Corporation), 1 μl pfu, dH₂O, 31 μl, were mixed to a total of 50 μl. After allowing this mixed solution at 95° C. for 5 minutes, reaction was done in two cycles of which one cycle consisted of (94° C. for 30 seconds), (55° C. for 30 seconds), and (72° C. for 60 seconds) and further, the reaction was repeated in 20 cycles of which one cycle consisted of (94° C. for 30 seconds), (58° C. for 30 seconds), and (72° C. for 60 seconds). The obtained PCR product was incorporated in PBS-SK (+) (STRATAGENE) at its XhoI-XbaI site to prepare pBS-bcl2.

Employing pBS-bcl2 and one or more kinds of various antisense oligonucleotides for variation introduction that were chemically synthesized separately (Oligo-1 to -9: SEQ ID NOS:11-19), variation was introduced at one or two positions of the Bcl-2 gene with an Altered Sites II-Ex1 in vitro Mutagenesis System (Promega Corporation) according to the procedure described below.

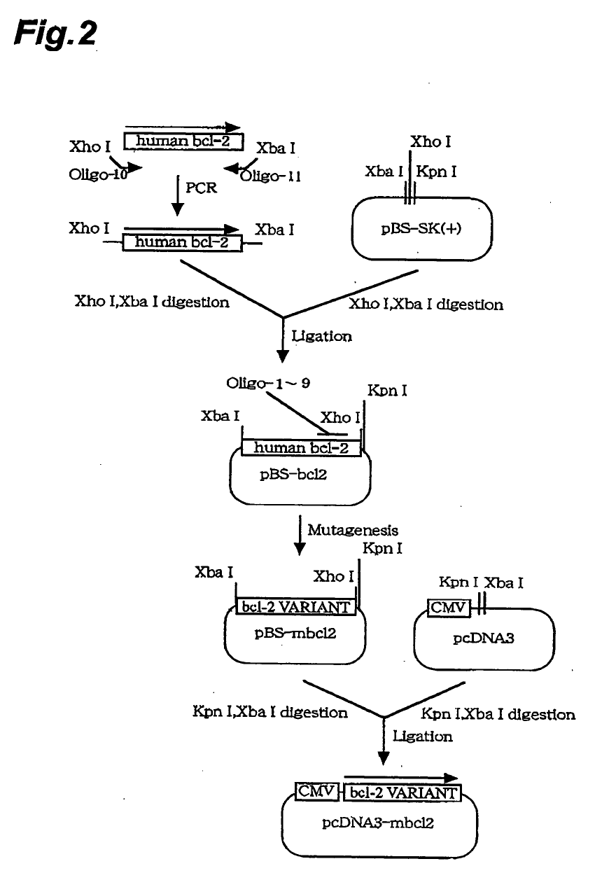

pBS-bcl2 (200 ng), 2 μl, Ampicillin Repair Oligonucleotide (0.25 μM: kit accessory available from Promega Corporation), 1 μl, Tetracycline Knockout Oligonucleotide (0.25 μM: kit accessory available from Promega Corporation.), 1 μl, an oligonucleotide for variation introduction (1.25 μM), 1 μl, Annealing 10×buffer (kit accessory available from Promega Corporation.), 2 μl, dH₂O, 13 μl, were mixed to a total of total 20 μl. After allowing this mixed solution at 95° C. for 5 minutes and then at 75° C. for 6 minutes, temperature was lowered to 45° C. at a rate of 1° C. per second. Subsequently, reaction was stopped at 37° C. To this reaction solution were mixed, Synthesis 10×buffer (kit accessory available from Promega Corporation.), 3 μl, T4 DNA polymerase (5-10 units: kit accessory available from Promega Corporation.), 1 μl, T4 DNA ligase (1-3 units: kit accessory available from Promega Corporation.), dH₂O, 5 μl, making a total of 30 μl. This mixed solution was further allowed to react at 37° C. for 90 minutes, whereby there were obtained 12 kinds in total of Bcl-2 variant DNAs corresponding to one or two kinds of antisense oligonucleotides of the variation type (e.g., Oligo-1 to -9). The Bcl-2 variant DNA was incorporated in pcDNA3 (Invitrogen Corporation) carrying a cytomegalovirus promoter capable of transcription in animal cells and finally, viral expression vectors having 12 kinds of Bcl-2 variant genes incorporated were constructed. See FIG. 2.

### Example 2

### Variation Gene Transfer to Cells

The calcium phosphate method was used in the procedure described below to conduct gene transfer into a BHK cell (syrian hamster kidney origin) with the following genes; the 12 kinds of Bcl-2 variant genes constructed in Example 1, the wild type Bcl-2 gene (as positive control), and pcDNA3 (as negative control)

BHK cells were seeded on a cover slip (18 mm: Matsunami Glass Co. Ltd.) to provide a level of 0.8-1.6×10⁴. Culturing was done under the conditions of 37° C. and 5% CO₂ overnight. After culturing, the culture medium was changed once and the culturing was done under the conditions of 37° C. and 5% CO₂ for additional 2-4 hours.

The gene/calcium phosphate solution was prepared according to the following method. To 30 μl of a calcium chloride solution (0.2 M CaCl2, 50 mM Hepes), were added the wild type Bcl-2, 4 μl, pcDNA3 (GFP), and the 12 kinds of Bcl-2 variant genes (each 2 μg). Further, to the solution was added dropwise 30 μl of 2×HBS (50 mm Hepes, 280 mM NaCl, 10 mM KCl, 1.5 mM Na₂HPO₄.12H₂O, 12 mM glucose, 0.1×PBS) under shaking. It was left at room temperature for 15 minutes, and 350 μl of medium was added last to prepare gene/calcium phosphate solutions. The prepared gene/calcium phosphate solution was added to the cells. After culturing for 4 hours, the cells were washed and the culture medium was changed. Further, after 16 hours' culturing, 4 mM of Dexamethazone (Sigma Company Ltd.) was added to the cells, inducing apoptosis. Furthermore, the cells were fixed with 3% formaldehyde after 8 hours' culturing and were used in the test that follows.

### Example 3

### Comparison of Apoptosis Inhibitory Effects by Nuclear Staining

The cells obtained in Example 2 and fixed with 3% formaldehyde, for which apoptosis was induced, were stained with 10 mg/ml of Hoechst33258 (Molecular Probe Inc.) for one minute and were observed under a microscope.

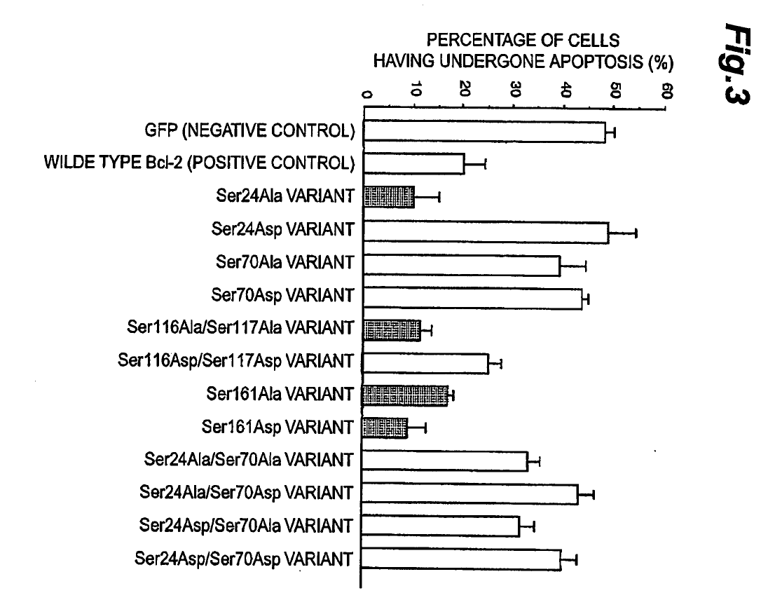

For the determination of apoptosis, the followings were observed and employed as indicators: (1) the distinct contraction of cytoskeleton; (2) the concentration of nucleus and increased staining; (3) round-up and blebbing of cell; (4) fragmentation of nucleus. (Wyllie et al., Int. Rev. Cytol. 68, 251 (1980).) If any one of (1) to (4) is applicable, the cell will be judged to be positive, and the number of cells is shown as a percentage of 1500 cells. The results obtained are shown in FIG. 3. Out of 12 kinds of Bcl-2 variants with variation introduced finally, the apoptosis inhibitory activity was significantly enhanced in the Ser24Ala variant, the Ser116Ala/Ser117Ala variant, the Ser161Ala variant, and the Ser161Asp variant, when compared with the wild type Bcl-2 which was positive control.

In particular, the result that the Ser24Ala variant derivable from substitution of the 24th serine in its BH4 domain by alanine strongly inhibit apoptosis is consistent with the fact that the particular site is susceptible to dephosphorylation by calcineurin.

### INDUSTRIAL APPLICABILITY

As described above, the polynucleotides and the polypeptides according to this invention are variants of the Bcl-2 gene, which is a protooncogene , and Bcl-2 variant proteins possessing apoptosis inhibitory activity, respectively; and said proteins are provided with significantly enhanced apoptosis inhibitory activity as compared with the wild type Bcl-2 protein.

Accordingly, the polypeptides of this invention are useful for the treatment of various disorders for which the apoptosis of cells is responsible.

Furthermore, because the polynucleotides of this invention can be the genes encoding the polypeptides possessing the apoptosis inhibitory activity, they will be useful for the treatment of various disorders for which the apoptosis of cells is responsible when applied in gene therapy.

1. A polypeptide comprising an amino acid sequence derivable from the substitution of at least one serine by alanine or asparagic acid in the amino sequence set forth in SEQ ID NO:1 in the Sequence Listing, said polypeptide possessing apoptosis inhibitory activity.

2. A polypeptide comprising an amino acid sequence derivable from the substitution of at least one serine by alanine or asparagic acid in the amino sequence set forth in SEQ ID NO:1 in the Sequence Listing, said polypeptide possessing apoptosis inhibitory activity substantially higher than that of the wild type Bcl-2 protein.

3. The polypeptide according to claim 2, wherein the serine is at least one selected from the 24th, the 116th, the 117th, or the 161st residue in the amino acid sequence.

4. The polypeptide according to claim 3, wherein the 24th, the 116th, and/or the 117th serine is substituted by alanine, and/or the 161st serine is substituted by alanine or asparagic acid.

5. A polypeptide comprising the amino acid sequence set forth in SEQ ID NO:2 in the Sequence Listing.

6. A polypeptide comprising the amino acid sequence set forth in SEQ ID NO:3 in the Sequence Listing.

7. A polypeptide comprising the amino acid sequence set forth in SEQ ID NO:4 in the Sequence Listing.

8. A polypeptide comprising the amino acid sequence set forth in SEQ ID NO:5 in the Sequence Listing.

9. A gene encoding the polypeptide according to any one of claims 1-8.

10. A polynucleotide comprising the nucleotide sequence set forth in SEQ ID NO:7 in the Sequence Listing.

11. A polynucleotide comprising the nucleotide sequence set forth in SEQ ID NO:8 in the Sequence Listing.

12. A polynucleotide comprising the nucleotide sequence set forth in SEQ ID NO:9 in the Sequence Listing.

13. A polynucleotide comprising the nucleotide sequence set forth in SEQ ID NO:10 in the Sequence Listing.

14. A pharmaceutical composition comprising the polypeptide according to any one of claims 1-8, a combination thereof, or a partial peptide thereof, together with a pharmaceutically acceptable carrier or diluent.

15. A viral vector having the gene according to claim 9 incorporated therein in the state capable of S being expressed.

16. A composition for gene therapy comprising the polynucleotide according to any one of claims 10-13 and a pharmaceutically acceptable carrier or diluent.


SEQUENCE LISTING (21 sequences)

SEQ ID NO: 1 - protein, 239 aa, Homo sapiens [sequence omitted]

SEQ ID NO: 2 - protein, 239 aa, Artificial Sequence
  Description of Artificial Sequence Synthetic modified Bcl-2 protein
MAHAGRTGYDNREIVMKYIHYKLAQRGYEWDAGDVGAAPPGAAPAPGIFSSQPGHTPHPAASRDPVARTSPLQTPAAPGAAAGPALSPVPPVVHLALRQAGDDFSRRYRGDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLSHK

SEQ ID NO: 3 - protein, 239 aa, Artificial Sequence
  Description of Artificial Sequence Synthetic modified Bcl-2 protein
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLALRQAGDDFSRRYRGDFAEMAAQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLSHK

SEQ ID NO: 4 - protein, 239 aa, Artificial Sequence
  Description of Artificial Sequence Synthetic modified Bcl-2 protein
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLALRQAGDDFSRRYRGDFAEMSSQLHLTPFTARGRFATVVEELFRDGVNWGRIVAFFEFGGVMCVEAVNREMSPLVDNIALWMTEYLNRHLHTWIQDNGGWDAFVELYGPSMRPLFDFSWLSLKTLLSLALVGACITLGAYLSHK

SEQ ID NO: 5 - protein, 239 aa, Artificial Sequence
  Description of Artificial Sequence Synthetic modified Bcl-2 protein
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLALRQAGDDFSRRYRGDFAEMSSQLHLTPFTARGRFATVVEELFRDGVNWGRIVAFFEFGGVMCVEDVNREMSPLVDNIALWMTEYLNRHLHTWIQDNGGWDAFVELYGPSMRPLFDFSWLSLKTLLSLALVGACITLGAYLSHK

SEQ ID NO: 6 - DNA, 720 bp, Homo sapiens
atggcgcacgctgggagaacggggtacgacaaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgcgacccggtcgccaggacctcgccgctgcagaccccggctgcccccggcgccgccgcggggcctgcgctcagcccggtgccacctgtggtccacctggccctccgccaagccggcgacgacttctcccgccgctaccgcggcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgagccacaagtga

SEQ ID NO: 7 - DNA, 720 bp, Artificial Sequence
  Description of Artificial Sequence Synthetic nucleotide sequence encoding modified Bcl-2 protein
atggcgcacgctgggagaacggggtacgacaaccgggagatagtgatgaagtacatccattataagctggcgcagaggggctacgagtgggatgcgggagatgtgggcgccgcgcccccgggggccgcccccgcaccgggcatcttctcctcccagcccgggcacacgccccatccagccgcatcccgcgacccggtcgccaggacctcgccgctgcagaccccggctgcccccggcgccgccgcggggcctgcgctcagcccggtgccacctgtggtccacctggccctccgccaagccggcgacgacttctcccgccgctaccgcggcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgagccacaagtga

SEQ ID NO: 8 - DNA, 720 bp, Artificial Sequence
  Description of Artificial Sequence Synthetic nucleotide sequence encoding modified Bcl-2 protein
atggcgcacgctgggagaacggggtacgacaaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgcgacccggtcgccaggacctcgccgctgcagaccccggctgcccccggcgccgccgcggggcctgcgctcagcccggtgccacctgtggtccacctggccctccgccaagccggcgacgacttctcccgccgctaccgcggcgacttcgccgagatggccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgagccacaagtga

SEQ ID NO: 9 - DNA, 720 bp, Artificial Sequence
  Description of Artificial Sequence Synthetic nucleotide sequence encoding modified Bcl-2 protein
atggcgcacgctgggagaacggggtacgacaaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgcgacccggtcgccaggacctcgccgctgcagaccccggctgcccccggcgccgccgcggggcctgcgctcagcccggtgccacctgtggtccacctggccctccgccaagccggcgacgacttctcccgccgctaccgcggcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggaggccgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgagccacaagtga

SEQ ID NO: 10 - DNA, 720 bp, Artificial Sequence
  Description of Artificial Sequence Synthetic nucleotide sequence encoding modified Bcl-2 protein
atggcgcacgctgggagaacggggtacgacaaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgcgacccggtcgccaggacctcgccgctgcagaccccggctgcccccggcgccgccgcggggcctgcgctcagcccggtgccacctgtggtccacctggccctccgccaagccggcgacgacttctcccgccgctaccgcggcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggaggacgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgagccacaagtga

SEQ ID NO: 11 - DNA, 27 bp, Artificial Sequence
  Description of Artificial Sequence Primer
gtagccggtctgcgccagcttataatg

SEQ ID NO: 12 - DNA, 27 bp, Artificial Sequence
  Description of Artificial Sequence Primer
gtagccggtctggtccagcttataatg

SEQ ID NO: 13 - DNA, 27 bp, Artificial Sequence
  Description of Artificial Sequence Primer
ggtcttgcagcggcgcggtcctggcgc

SEQ ID NO: 14 - DNA, 27 bp, Artificial Sequence
  Description of Artificial Sequence Primer
ggtctgcagcgggtcggtcctggcgac

SEQ ID NO: 15 - DNA, 27 bp, Artificial Sequence
  Description of Artificial Sequence Primer
gtgcagctggctggccatctcggcgaa

SEQ ID NO: 16 - DNA, 27 bp, Artificial Sequence
  Description of Artificial Sequence Primer
gtgcagctggctgaccatctcggcgaa

SEQ ID NO: 17 - DNA, 27 bp, Artificial Sequence
  Description of Artificial Sequence Primer
gtgcagctgggcggccatctcggcgaa

SEQ ID NO: 18 - DNA, 27 bp, Artificial Sequence
  Description of Artificial Sequence Primer
ctcccggttgacggcctccacacacat

SEQ ID NO: 19 - DNA, 27 bp, Artificial Sequence
  Description of Artificial Sequence Primer
ctcccggttgacgacctccacacacat

SEQ ID NO: 20 - DNA, 36 bp, Artificial Sequence
  Description of Artificial Sequence Primer
ccgctcgagatggcgcacgctgggagaacggggtac

SEQ ID NO: 21 - DNA, 32 bp, Artificial Sequence
  Description of Artificial Sequence Primer
gctctagatcacttgtggctcagataggcacc